SEQUENCE LISTING (8 sequences)

SEQ ID NO: 1 - DNA, 1626 bp, Bifidobacterium adolescentis
  source 1..1626: mol_type = genomic DNA
gaaggagatatacaaatggctagcagaggatcgcatcaccatcaccatcacggggccggagacctgatgaaaaataaggtacaacttattacctacgcagaccgtcttggcgacggaacgatcaagtcgatgaccgatatcttacgtacacgctttgatggtgtgtatgatggtgtgcacattctgccattcttcacgccgtttgacggggcagacgcaggctttgatcccatcgatcatacaaaggtagacgaacgtcttggttcttgggacgacgtcgcagaattaagcaagacgcacaacatcatggtcgatgcgatcgtaaatcacatgtcgtgggagtccaaacagttccaggacgttttggcaaaaggtgaagaatccgagtactatccgatgtttttaacgatgtcgtctgtatttcccaacggggctacggaagaggaccttgccggaatttaccgccctcgtcctggtctgcccttcacccattacaaattcgctggcaagactcgtcttgtctgggtctcgttcacgccgcaacaggtggacatcgacactgacagcgataagggatgggagtacttgatgtcaatttttgatcaaatggcggcctcgcacgtctcgtacattcgccttgatgcagtcggttatggtgcgaaggaggctggcacctcctgctttatgactccgaagacttttaagctgatcagccgcttacgtgaggagggtgtgaagcgtggactggaaattttaattgaggtccactcttactataaaaaacaagtggagatcgcgtcgaaggtcgatcgtgtttacgactttgcattaccacccttattattgcacgccttatctacagggcatgtggaaccggtggcgcattggacagacatccgcccgaataatgctgttacagtcctggatacacatgatggaatcggcgtcatcgacattgggtcggatcagttggatcgttcgttgaagggtcttgttcccgacgaagacgtcgataatttagtgaatacgattcacgccaatactcatggagaatcggaggctgctacaggtgccgcagctagtaatttagatctgtaccaggtcaactcaacctattactctgcattaggctgtaatgaccaacactacattgctgcacgtgctgtgcagttttttttaccgggggttccacaggtttactacgtgggcgcattagctggaaagaacgatatggagttgcttaataagaccaacaacggacgcgatatcaatcgccactattactccacggctgagatcgacgagaatttgaagcgtcccgtagtcaaggcacttaatgcattagcgaagtttcgcaatgagttagatgcatttgatggaacattcagctacactacacccactgatacatcgatttcgtttacttggcgtggggaaactagcgaggccacactgacgttcgagcctaaacgcggcttgggcgtagataatacaacacccgtcgcgatgttagagtggcatgatagcgcaggagaccatcgtagtgatgacctgatcgccaatccgccggtcgttgcgtaaaccatggtctcccatcacctaagcttcgatagcagctacgttaac

SEQ ID NO: 2 - DNA, 939 bp, unidentified
  source 1..939: mol_type = genomic DNA
gaaggagatatacaaatggctagcagaggatcgcatcaccatcaccatcacggggccggagacctgatgattaagaagtgccttttcccggcggccggttacggaacccgttttcttccgattaccaagactatccccaaggaaatgctgccaatcgtagacaagcctcttatccagtatgccgtggaagaagcaatggaggcaggctgtgaagtgatggctattgtcaccggacgcaacaagcgtagtttggaggattacttcgacacgtcgtatgaaatcgagcatcagatccaaggtacgaacaaagagaatgcccttaagtcgatccgcaacattatcgagaaatgttgcttctcgtacgtccgtcaaaaacagatgaaagggctgggacatgcaatcttgacgggagaggctctgatcggaaatgaaccgttcgcagttatcttagcagacgatttgtgtatctcacatgatcatccttcagtgctgaaacaaatgacttcgttataccaaaagtatcaatgcagcatcgtagccatcgaggaagtcgcccttgaagaggtgtccaagtatggagttatccgcggagagtggttagaagaaggggtctacgagattaaggatatggtcgagaagcccaatcaagaagatgcaccatcaaatttagccgttatcggccgctatatcttaacgccggatatcttcgagatcctgtcagagacgaagcctggtaaaaataatgagattcagattacagatgctttgcgtactcaggctaagcgtaaacgcattattgcatatcaatttaagggcaaacgctacgattgtggcagcgttgagggatacattgaggcgtctaacgcctattacaaaaagcgcttgcttgaataaaccatggtctcccatcacctaagcttcgatagcagctacgttaac

SEQ ID NO: 3 - DNA, 771 bp, unidentified
  source 1..771: mol_type = genomic DNA
gaaggagatatacaaatggctagcagaggatcgcatcaccatcaccatcacggggccggagacctgggatctcacatgcgtcagccaattgcactgatttctgtccacggcgatccggcggcagacgtaggccatgagtccgcgggtggacaaaacatttatgtgcgccaattgggcgaagcactggccgcagctggttggcatgtggacatgtttacacgcaagaccgacccaaatgatcctgacgtgattgaacatagcccccactgccgcacgattcgtttgcaagccggtccgcttacgtacattcctcgcgaaaaacttttcgagactctgcccaagttcgtagaagcctttaaggcgtatcacgctaagtatgggtatcctctgatccatacgaactactggttgagtggctgggtaggctggcaattgcgccagcaattcaatttccagtggttgcacacgtatcattccttgggtgttgttaagtatcaggtcgcttccgaacaggcccagcgtgacgaaacacgtttaatggtagaaaaagctatcttggaaaatgcagactgtgtcatcgttacttcaccacaagaggaagcatatttgcgtcgctgggtatcaaaggccggtcagactcgtttgatcccttgtggcactaactgggaagccatcgctttgcaaatgggtcaattatatcgccaattgttcgccgcatcactttaaaccatggtctcccatcacctaagcttcgatagcagctacgttaac

SEQ ID NO: 4 - DNA, 1374 bp, unidentified
  source 1..1374: mol_type = genomic DNA
gaaggagatatacaaatggctagcagaggatcgcatcaccatcaccatcacggggccggagacctgatggataagttgagcggatctgcacgcttaatgattgtctccgatcttgaccatacaatggtggaccaccacgatgaagagaacttatctttgttgcgcttcggcgctctgtgggagtccgtctactgtgaagatagcctgctggtttttagtaccgggcgttcgcccacgttgtacaaagagttacgcaaagagaaacccatgttgaccccagacattaccatcatgtctgtagggacagaaatcacctatggcgaagctatggttcctgatgacggctgggaagaatacttaaacaataaatgggaccgcaatatcgtggtagctgagaccgtctctttcagcgaattaaaattacagccagaaacggaacaacgtccacataaggtatctttttttgtggacaagaaaaatgcccaggaagtaatcaagagcgtagctgagcgtctggacaaatgcgggctggacgcaaaaattatctattccgggggccaggatctggacatcctgccccagggggctggcaaggggcaagcccttgcgtatttacttgaaaaactgagttcctgcggcaaaccccccaacaacacattggtttgcggggattctgggaacgatgcagaattgttttccatccccggtgtccacggagtgatggtctccaatgcacaagaagagttgctgcagtggtacacggaaaacgcgaaggataatccaaagatcatccactcaaacgaacgctgtgcggcaggaatcattcaagcgattggacatttcaagttgggtccgaatatcagtccacgtgatcttcagtttccttatgcaaaagaggcgagcttcaagccgacagacgccgtagtaaagttttatgtgctgtacgaaaagtggcgtcgtgctgaggtcccaaaaagcgatagtgtcattaaatactttaaaaacatcacacacgcaaacggagtaacaattcatccggctggcttagaattatcgttgcacgcctcgattgacgcacttggttcttgctacggtgacaagcaaggccgtaaatatcgtgcctgggtagatcgtttatttattacccagaccggatccgacagttgggtagggcgcttcgacctttgggagagcgagggggatgtgcgcgtctgcagcttatcatcattggcgttaatcttaaaggccgagagcccggagggatttgtcttaacacatattcagaaaacttggctgaacggatactccagcggtgtagaacaggcctaaaccatggtctcccatcacctaagcttcgatagcagctacgttaac

SEQ ID NO: 5 - protein, 521 aa, Bifidobacterium adolescentis
MASRGSHHHHHHGAGDLMKNKVQLITYADRLGDGTIKSMTDILRTRFDGVYDGVHILPFFTPFDGADAGFDPIDHTKVDERLGSWDDVAELSKTHNIMVDAIVNHMSWESKQFQDVLAKGEESEYYPMFLTMSSVFPNGATEEDLAGIYRPRPGLPFTHYKFAGKTRLVWVSFTPQQVDIDTDSDKGWEYLMSIFDQMAASHVSYIRLDAVGYGAKEAGTSCFMTPKTFKLISRLREEGVKRGLEILIEVHSYYKKQVEIASKVDRVYDFALPPLLLHALSTGHVEPVAHWTDIRPNNAVTVLDTHDGIGVIDIGSDQLDRSLKGLVPDEDVDNLVNTIHANTHGESEAATGAAASNLDLYQVNSTYYSALGCNDQHYIAARAVQFFLPGVPQVYYVGALAGKNDMELLNKTNNGRDINRHYYSTAEIDENLKRPVVKALNALAKFRNELDAFDGTFSYTTPTDTSISFTWRGETSEATLTFEPKRGLGVDNTTPVAMLEWHDSAGDHRSDDLIANPPVVA

SEQ ID NO: 6 - protein, 292 aa, unidentified
MASRGSHHHHHHGAGDLMIKKCLFPAAGYGTRFLPITKTIPKEMLPIVDKPLIQYAVEEAMEAGCEVMAIVTGRNKRSLEDYFDTSYEIEHQIQGTNKENALKSIRNIIEKCCFSYVRQKQMKGLGHAILTGEALIGNEPFAVILADDLCISHDHPSVLKQMTSLYQKYQCSIVAIEEVALEEVSKYGVIRGEWLEEGVYEIKDMVEKPNQEDAPSNLAVIGRYILTPDIFEILSETKPGKNNEIQITDALRTQAKRKRIIAYQFKGKRYDCGSVEGYIEASNAYYKKRLLE

SEQ ID NO: 7 - protein, 236 aa, unidentified
MASRGSHHHHHHGAGDLGSHMRQPIALISVHGDPAADVGHESAGGQNIYVRQLGEALAAAGWHVDMFTRKTDPNDPDVIEHSPHCRTIRLQAGPLTYIPREKLFETLPKFVEAFKAYHAKYGYPLIHTNYWLSGWVGWQLRQQFNFQWLHTYHSLGVVKYQVASEQAQRDETRLMVEKAILENADCVIVTSPQEEAYLRRWVSKAGQTRLIPCGTNWEAIALQMGQLYRQLFAASL

SEQ ID NO: 8 - protein, 437 aa, unidentified
MASRGSHHHHHHGAGDLMDKLSGSARLMIVSDLDHTMVDHHDEENLSLLRFGALWESVYCEDSLLVFSTGRSPTLYKELRKEKPMLTPDITIMSVGTEITYGEAMVPDDGWEEYLNNKWDRNIVVAETVSFSELKLQPETEQRPHKVSFFVDKKNAQEVIKSVAERLDKCGLDAKIIYSGGQDLDILPQGAGKGQALAYLLEKLSSCGKPPNNTLVCGDSGNDAELFSIPGVHGVMVSNAQEELLQWYTENAKDNPKIIHSNERCAAGIIQAIGHFKLGPNISPRDLQFPYAKEASFKPTDAVVKFYVLYEKWRRAEVPKSDSVIKYFKNITHANGVTIHPAGLELSLHASIDALGSCYGDKQGRKYRAWVDRLFITQTGSDSWVGRFDLWESEGDVRVCSLSSLALILKAESPEGFVLTHIQKTWLNGYSSGVEQA

(19) United States
(12) Patent Application Publication
(10) Pub. No.: US 2026/0337650 A1
(43) Pub. Date: Oct. 29, 2026

(54) DISACCHARIDE WITH METABOLIC BENEFITS AND MONOSACCHARIDE MIXTURES

(71) Applicant: Nutrishus Brands Inc., Atlanta, GA (US)

(72) Inventors: Steven E. HANLEY, Hilton Head, SC (US); Richard J. JOHNSON, Centennial, CO (US); Steven BENNER, Gainesville, FL (US); Roberto LAOS, Gainesville, FL (US); Yubing LI, Gainesville, FL (US)

(21) Appl. No.: 19/449,319

(22) Filed: Jan. 14, 2026

Related U.S. Application Data
(63) Continuation-in-part of application No. PCT/US2024/044289, filed on Aug. 28, 2024.
(60) Provisional application No. 63/535,942, filed on Aug. 31, 2023.
(60) Provisional application No. 63/745,056, filed on Jan. 14, 2025.

(51) Int. Cl. C12P 19/12; A23L 2/60; A23L 27/30; C12N 9/10; C12N 9/12; C12N 9/16; C12N 9/26; C12N 9/92; C12P 19/14; C12P 19/18; C12P 19/24

(52) U.S. Cl. CPC C12P 19/12; A23L 2/60; A23L 27/33; C12N 9/1051; C12N 9/1066; C12N 9/1205; C12N 9/1229; C12N 9/1241; C12N 9/16; C12N 9/2411; C12N 9/92; C12P 19/14; C12P 19/18; C12P 19/24; C12Y 204/01001; C12Y 204/01007; C12Y 204/01014; C12Y 207/01001; C12Y 207/04001; C12Y 207/07009; C12Y 301/03024; C12Y 302/01; C12Y 501/03; C12Y 503/01009; C12Y 503/01018; C12Y 504/02002

(57) ABSTRACT

Disclosed herein are strategies for making α-D-glucopyranosyl-β-allulofuranoside (a disaccharide containing glucose and allulose moieties) also referred to herein as SUPERCROSE™. Also disclosed are allulose-glucose mixtures lacking fructose for enhancing flavor of food and beverages.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 28, 2024, is named “16786-002PC0.xml” and is 13,906 bytes in size. The sequence listing contained in this .XML file is part of the specification and is hereby incorporated by reference herein in its entirety.

### BACKGROUND

Excessive ingestion of fructose, primarily in the form of added sugars, such as high fructose corn syrup (HFCS) and sucrose, may have a role in the epidemic of obesity and diabetes (Malik V S, Hu F B. (2022) The role of sugar-sweetened beverages in the global epidemics of obesity and chronic diseases. Nat Rev Endocrinol 18, 205-218). Administering high concentrations of fructose to both animals and humans can induce features of metabolic syndrome, including insulin resistance, hypertriglyceridemia, low HDL cholesterol, fatty liver, and elevated blood pressure (Ishimoto T, et al. (2012) Opposing effects of fructokinase C and A isoforms on fructose-induced metabolic syndrome in mice. Proc Natl Acad Sci 109, 4320-4325) and Perez-Pozo S. E., et al. (2010) Excessive fructose intake induces the features of metabolic syndrome in healthy adult men: role of uric acid in the hypertensive response. Int J. Obes 34, 454-461)). Similar effects can be observed by administering drinks rich in sucrose or high fructose corn syrup, both which contain fructose and glucose. These types of studies, as well as experiments in animal models, have clearly demonstrated that the effects of fructose to induce metabolic syndrome are independent of excessive energy intake (Andres-Hemando A, et al. (2020). Deletion of Fructokinase in the Liver or in the Intestine Reveals Differential Effects on Sugar-Induced Metabolic Dysfunction. Cell Metab. 32, 117-127).

The ingestion of sucrose (table sugar) also causes a feeling of pleasantness as a consequence of stimulating dopamine in the brain (Avena N M, Rada P, Hoebel B G. (2008) Evidence for sugar addiction: behavioral and neurochemical effects of intermittent, excessive sugar intake. Neurosci Biobehav Rev. 32, 20-39)). Indeed, repeated ingestion of sugar in mice can lead to a craving or addiction syndrome. These animals develop features similar to that observed with drug addiction, and will show signs of anxiety or withdrawal following elimination of sugar from the diet or upon the administration of naloxone. This craving for sugar is linked with fructose metabolism and may be important in the pathogenesis of obesity, attention deficit hyperactivity disorder, and even dementia (Andres-Hernando A, et al MA. (2020) Sugar causes obesity and metabolic syndrome in mice independently of sweet taste. Am J Physiol Endocrinol Metab. 319, E276-E290).

Allulose (also known as D-psicose) is a monosaccharide that occurs in nature and is produced in trace amounts. Allulose is a C-3 epimer of D-fructose with a sweetness that is 70% that of sucrose. Unlike fructose or sucrose, allulose is minimally metabolized in the human body and has a caloric value of 0.42 kcal/g. Allulose is also distinct from fructose as intake of allulose inhibits the formation of body fat (Matsuo, T. et. al., (2001) Dietary D-picose, a C-3 epimer of D-fructose, suppresses the activity of hepatic lipogenic enzymes in rats. Asia Pac. J. Clin. Nutr., 10, 233-237); Matsuo, T. and K. Izumori, (2004) Asia Pac. J. Clin. Nutr., 13, S127)) and reduces body fat in humans (Han Y, et al. (2018) A Preliminary Study for Evaluating the Dose-Dependent Effect of d-Allulose for Fat Mass Reduction in Adult Humans: A Randomized, Double-Blind, Placebo-Controlled Trial. Nutrients. 10, 160). Allulose also does not raise blood sugar but rather reduces the glycemic response to carbohydrates including sucrose (F. Au-Yeung et al. (2023) Comparison of postprandial glycemic and insulinemic response of allulose when consumed alone or when added to sucrose: A randomized controlled trial. J Functional Foods. 105, 105569). This makes allulose an attractive substitute for sucrose or fructose.

Disaccharides formed between aldohexoses (such as glucose) and/or ketohexoses (such as fructose) are known throughout nature, where they have numerous uses. For example, isomaltulose is a naturally occurring disaccharide of glucose (at its carbon-1 position) and fructose (at its sixth carbon position), and is used in low glycemic foods and beverages (Holub, I., et al. (2010) Novel findings on the metabolic effects of the low glycemic carbohydrate isomaltulose (Palatinose). British J. Nutrition 103, 1730-1737). It is similar to sucrose except that the fructose and glucose are bonded differently and this leads to differences in absorption and other characteristics. Provided below is a list of known primary sugar disaccharides, monosaccharides and rare sugars:

### Disaccharides:

1. Sucrose—Commonly known as table sugar, composed of glucose and fructose.
2. Lactose—Found in milk, composed of glucose and galactose.
3. Maltose—Known as malt sugar, composed of two glucose molecules.
4. Trehalose—Composed of two glucose molecules, found in fungi and insects.
5. Celloblose—A hydrolysis product of cellulose, composed of two glucose molecules.

### Monosaccharides:

1. Glucose—A primary energy source for cells.
2. Fructose—Found in fruits, honey, and root vegetables.
3. Galactose—A component of lactose, found in dairy products.
4. Ribose—A component of RNA.
5. Mannose—Found in some fruits and vegetables, plays a role in human metabolism.
6. Xylose—Found in wood and plant cells.
7. Arabinose—Common in plants, especially in the hemicellulose component.

### Rare Sugars:

1. Allulose (D-Psicose)—A low-calorie sweetener, naturally found in small quantities in fruits.
2. Allose—A rare sugar with potential therapeutic properties.
3. Tagatose—A low-calorie sweetener, similar in texture and taste to sucrose.
4. Sorbose—Found in the synthesis of Vitamin C.
5. Mannitol—Used as a sugar substitute in diabetic-friendly foods.
6. Xylitol—A sugar alcohol used as a sugar substitute.
7. Rhamnose—Found in plants, often used in cosmetics.

Glucose can also combine with ketohexoses other than fructose to form disaccharides. Many of these alternative ketohexoses are available through enzymatic processes. These include D-tagatose, D-sorbose, D-allulose, L-fructose, L-allulose, and L-tagatose, (Izumori, K. (2006) A strategy for bioproduction of all hexoses. J. Biotechnol. 124, 717-722). Disaccharides with these monosaccharides combined to glucose have been discussed (Morimoto, K., Yoshihara, A., Furumoto, T., Takata, G. (2015) Production and application of a rare disaccharide using sucrose phosphorylase from Leuconostoc mesenteroides. J. Bioscience Bioengineering 119, 652-656, incorporated in its entirety herein by reference, together with its cited literature).

The complexity of their molecular structures, in particular their multiple hydroxyl groups with nearly equivalent reactivity, means that mono- and disaccharides are difficult to make by non-enzymatic “chemical” synthetic processes, and when they are, these synthetic processes are seldom inexpensive enough to support commercial production. Barrett et al. provides an example showing the complexity of a “chemical” synthesis of sucrose (Barrett A G, Melcher L M, Bezuidenhoudt B. C., Redox glycosidation: the use of Nozaki-Takai methylenylation in a highly stereoselective synthesis of sucrose. Carbohydr Res. 1992 Aug. 3; 232(2):259-72). Accordingly, most commercial disaccharides, notably sucrose, are isolated from plant sources.

Enzymatic processes for making disaccharides are preferred over ‘chemical’ syntheses. Morimoto, for example, made a disaccharide consisting of glucose and allulose. Nevertheless, the glucose-1-phosphate used in the process to make a disaccharide of glucose with allulose is much too expensive to be used at scale in a commercial process. Further, the process disclosed in Morimoto et al. (2015, id) that uses the less expensive sucrose as a starting material is reported to yield a product that is difficult to purify. In particular, it contained sucrose, which is known to have bad health effects.

It is noteworthy, that the hydrolysis of D-glucosyl-D-alluloside in humans would lead to the formation of D-glucose and D-allulose, which are both safe for human consumption.

### BRIEF DESCRIPTION OF THE DRAWINGS

FIG. 1. shows the chemical structure of SUPERCROSE™ (α-D-glucopyranosyl-(1→2)-β-D-allulofuranoside).

FIG. 2. provides a scheme of a general enzymatic method for producing SUPERCROSE™ utilizing starch as a source material.

FIG. 3 shows the ¹³C-NMR of SUPERCROSE™ produced using sucrose phosphorylase and ¹³C1-labeled glucose 1-phosphate. Shown is a ¹³CNMR spectrum of SUPERCROSE™ synthesized enzymatically. This reaction uses a labeled ¹³C-labeled glucose 1-phosphate. After overnight reaction, the peaks at 91.990 and 91.823 emerge. These peaks are equivalent to the ones in FIG. 4 that correspond to the C1 of the glucose moiety. The difference in ppm in comparison to FIG. 4 arises from the absence of the 0 ppm reference signal. Since this experiment has a carbon enriched in ¹³C, the signals are small, as the natural abundance of ¹³C is only 1%.

FIG. 4 is a ¹³CNMR spectrum of SUPERCROSE™ synthesized non-enzymatically. The synthesis used unlabeled precursors. The peaks have the same chemical shifts as reported by (Morimoto et al. (2015) Production and application of a rare disaccharide using sucrose phosphorylase from Leuconostoc mesenteroides. J. Bioscience Bioengineering 119, 652-656).

FIG. 5. Novel method to synthesize SUPERCROSE™ from starch using sucrose phosphate synthase. In addition to the net conversion of two glucose units in starch to one molecule of SUPERCROSE™, one molecule of UTP is converted to one molecule of UDP. This UDP is phosphorylated back to UTP in the route in FIG. 6.

FIG. 6 shows an enzymatic approach to regenerate UTP from UDP.

FIG. 7 provides a scheme showing two additional pathways to obtain allulose-6-phosphate that can be used for producing SUPERCROSE™. Both require ATP regeneration using the polyphosphate kinase system.

### DETAILED DESCRIPTION

### Definitions

In order to express an enzyme needed for the intermediate steps proposed to obtain SUPERCROSE™ well established methods known in the art are employed. For instance: synthetically made DNA can be cloned into an expression vector and introduced into a suitable strain of E. coli. The desired clone is verified by sequencing and introduced into another strain of E. coli suitable for expression. The methods used for extraction and purification are well known to trained scientists in the field.

In the method of the present invention, the technique of introducing a plasmid in a cell is well-known in the art. Examples in the art are abundant, for example, in Ausubel F. A., et al. ed. (1988), Current Protocols in Molecular Biology, Wiley, New York, N.Y.; Sambrook J, et al. (1987) Molecular Cloning: A Laboratory Manual, 2^{nd} Ed., Cold Spring Harbor Laboratory Press, Cold Spring Harbor, N.Y., and Bessatsu Jikken-igaku “ldenshidounyu & Hatsugen kaiseki jikkenhou”, Yodosha, 1997.

The term “glucose-1-phosphate” or α-D-1-glucose phosphate refers to glucose-1-phosphoric acid (C₆H₁₃O₉P) and a salt thereof. Glucose-1-phosphate may be any salt of glucose-1-phosphoric acid (C₆H₁₃O₉P). Specific examples of glucose-1-phosphate include disodium glucose-1-phosphate, dipotassium glucose-1-phosphate, and glucose-1-phosphoric acid (C₆H₁₃O₉P). In the present specification, glucose-1-phosphate whose chemical formula is not drawn in a parenthesis indicates glucose-1-phosphate in a wide sense, that is, glucose-1-phosphoric acid (C₆H₁₃O₉P) in a narrow sense and a salt thereof.

The term “subjecting” when used in the context of an enzymatic reaction means bringing a substrate(s) in contact with an enzyme under conditions (e.g. temperature, stoichiometry, buffer, medium, etc.) that allow for or promote the production of a desired product.

The term “α-glucan phosphorylase” refers to an enzyme having α-glucan phosphorylase activity. α-glucan phosphorylase is classified into EC2.4.1.1. α-glucan phosphorylase activity refers to the activity of catalyzing a reaction making glucose-1-phosphate and a partial degradation product of α-1,4-glucan from inorganic phosphoric acid (such as orthophosphate) and α-1,4-glucan, or a reverse reaction thereof. α-glucan phosphorylase is called phosphorylase, starch phosphorylase, glycogen phosphorylase, maltodextrin phosphorylase, and the like, in some cases. α-glucan phosphorylase can also catalyze a α-1,4-glucan synthesis reaction which is the reverse reaction of phosphorolysis. In which direction any particular reaction progresses depends on the amount of a substrate. In vivo, since the amount of inorganic phosphoric acid is large, the reaction of α-glucan phosphorylase proceeds towards the direction of phosphorolysis. When the amount of inorganic phosphoric acid is small, the reaction proceeds towards the synthesis of α-1,4-glucan.

In the present specification, the “α-glucan” refers to a saccharide containing D-glucose as a constituent unit and having at least two saccharide units or more of a saccharide unit linked with an α-1,4-glucoside bond. An α-glucan can be a linear, branched or cyclic molecule. A linear glucan has the same meaning as that of α-1,4-glucan. In a linear α-glucan, saccharide units are linked only with an α-1,4-glucoside bond. A α-glucan containing one or more α-1,6-glucoside bonds is a branched α-glucan. An α-glucan may contain a linear section to some extent. A linear α-glucan having no branching (such as amylose) is more preferable over those that are branched (amylopectin).

The term “starch” refers to starch and starch derivatives. Starch is a polymeric carbohydrate consisting of numerous glucose units joined by glycosidic bonds. Starch derivatives include any one of starch derivatives such as hydrolyzed starch, amylodextrine, maltodextrin, maltopolysaccharide or a mixture of two or more of these starch derivatives in any proportion.

In the present specification, the terms “sucrose phosphorylase” and “SP” are interchangeable unless otherwise specifically indicated, and means an enzyme having a sucrose phosphorylase activity. Sucrose phosphorylase is classified as EC. 2.4.1.7. Sucrose phosphorylases are also known as sucrose:phosphate D-glucosyltransferase and in the presence of inorganic phosphate (Pi) can degrade sucrose to glucose 1-phosphate and fructose. Like starch phosphorylase, it can also work in the reverse direction. Sucrose phosphorylases are also not fully specific for sucrose, but can donate the glucose 1-phosphate to other sugars to make novel disaccharides (see Desmet et al. Discovery, Application and Engineering. Int J Mol Sci. 2020 Apr. 5; 21(7):252). In certain examples, the natural sucrose phosphorylase can be derived from a bacterium selected from the group consisting of Streptococcus mutans, Streptococcus pneumoniae, Streptococcus sorbinus, Streptococcus mitis, Leuconostoc mesenteroides, Oenococcus oeni, Lactobacillus acidophilus and Listeria monocytogenes. In one embodiment, the natural sucrose phosphorylase can be derived from a bacterium selected from the group consisting of Streptococcus mutans, Streptococcus pneumoniae, Streptococcus sorbinus and Streptococcus mitis.

In one embodiment, the natural sucrose phosphorylase can be derived from Bifidobacterium adolescentis, Streptococcus mutans or Leuconostoc mesenteroides, among others.

The term “allulose-epimerase” or “epimerase” as used herein means a D-allulose-3-epimerase having a conversion activity of D-fructose to D-allulose. Examples of allulose-epimerases are set forth in Table 1.

The term glucose isomerase refers to a protein that has enzymatic activity to convert hexose units such as D-glucose whether as single hexose units or as part of an oligosaccharide into fructose. Isomerase which acts on hexose catalyzes “a reaction not causing a change in chemical formula C₆H₁₂O₆”. For example, aldose isomerase catalyzes “an intramolecular hydrogen transfer” and accelerates a redox reaction between C₁ and C₂. An enzyme “isomerase” is well known in this industry and examples of it include ketose 3-epimerase, xylose isomerase, and glucose isomerase. Technically, glucose isomerase is not a scientifically acknowledged name. The enzyme name “glucose isomerase” is not included in EC number registered in International Union of Biochemistry and Molecular Biology. D-xylose isomerase is its scientific name and glucose isomerase is only used in general as a commercial name. Accordingly, the term “glucose isomerase” is used interchangeably with other names of enzymes that are known to convert D-glucose to D-fructose, including for example xylose isomerase or L-rhamnose isomerase. Examples of glucose isomerase enzymes are provided in Table 1.

The term “amylase” refers to an enzyme that catalyzes the hydrolysis of starch into glucose or glucose dimers or polymers. In one example, the amylase is α-amylase. The amylase may also include a glucoamylase. Amylases can be used to break down both the amylose and amylopectin in starch, thereby facilitating the ability of starch phosphorylase to generate glucose-1-phosphate. The amylases can be derived from organisms known in the art. See for example, U.S. Pat. Pub. 20130274350, incorporated by reference.

The term “polyphosphate kinase 3” or “PPK3” refers to an enzyme that converts ADP to ATP in the presence of polyphosphates.

The term “allulose-6-phosphate 3-epimerase” refers to an enzyme that converts fructose-6-phosphate to allulose-6-phosphate.

The term “UDP-Glucose Pyrophosphorylase” refers to an enzyme that converts glucose-1-phosphate to UDP-glucose in the presence of UTP.

The term “phosphoglucomutase” or GSDXIV refers to an enzyme that converts glucose-1-phosphate to glucose-6-phosphate.

The term “phosphoglucoseisomerase” refers to an enzyme that converts glucose-6-phosphate to fructose-6-phosphate.

The term “sucrose phosphate synthase” refers to an enzyme that converts UDP-Glucose and allulose-6-phosphate to SUPERCROSE-6-phosphate.

The term “sucrose phosphate phosphatase” refers to an enzyme that converts SUPERCROSE-6-phosphate to SUPERCROSE™.

### Description of Embodiments

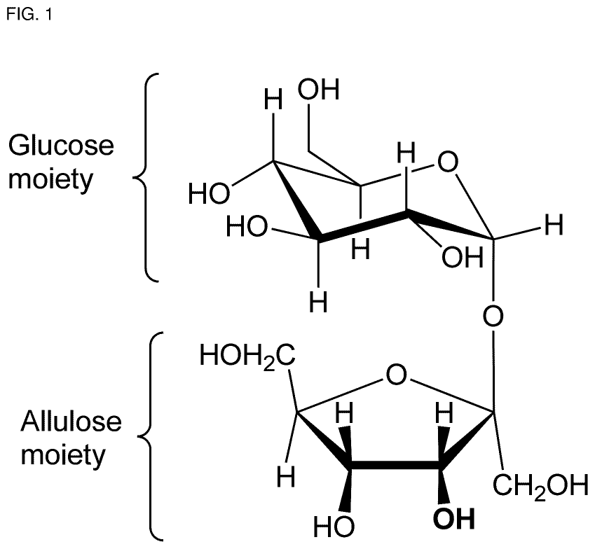

Disclosed herein are strategies for making α-D-glucopyranosyl-β-allulofuranoside (a disaccharide containing glucose and allulose moieties) henceforth called SUPERCROSE™. The chemical structure of SUPERCROSE™ is shown in FIG. 1. The strategies may have variations as are also described herein. This disaccharide harnesses the health benefits of allulose but also confers a sweetness distinct from an equal mix of glucose and allulose alone. A number of benefits associated with allulose and replacement of fructose and/or fructose containing disaccharides (e.g. sucrose) with allulose have been identified. We also disclose a mixture of the same two monosaccharides (glucose and allulose) that may also have additional benefits compared to either monosaccharide alone.

While allulose has beneficial metabolic effects when administered alone or in the presence of added sugars such as high fructose corn syrup or sucrose, there are some potential advantages of having a disaccharide with glucose and allulose. First, allulose is generally well tolerated, but when given at high doses alone it can cause some GI distress (Han Y, et al. (2018) Gastrointestinal Tolerance of D-Allulose in Healthy and Young Adults. A Non-Randomized Controlled Trial. Nutrients. 10, 160), similar to what has been observed with fructose (Skoog S M, Bharucha A E. (2004) Dietary fructose and gastrointestinal symptoms: a review. Am J Gastroenterol. 99, 2046-2050). The gastrointestinal side effects from fructose are markedly ameliorated when fructose is combined with glucose (Rumessen J J, Gudmand-Hoyer E. (1986) Absorption capacity of fructose in healthy adults. Comparison with sucrose and its constituent monosaccharides. Gut. 27, 1161-1168), and hence we predict better tolerability of allulose when it is combined with glucose as a disaccharide, thereby allowing higher concentrations of allulose to be tolerated. Importantly, this better tolerability is still expected to result in the blunting of the postprandial hyperglycemia from the glucose, similar to what is observed when allulose is administered with sucrose. The substitution of allulose for fructose will also reduce the amount of fructose ingested, which would not occur if allulose is given with sucrose (since sucrose is a disaccharide containing fructose and glucose). This is important since fructose is the key nutrient driving the metabolic response (Johnson R J, et al. (2023) The fructose survival hypothesis for obesity. Philos Trans R Soc Lond B Biol Sci. 378.20220230). In addition, an allulose-glucose disaccharide is likely to break down slower than a mixture of glucose and allulose, and slower absorption will translate into a less postprandial rise in glucose. Thus, a disaccharide containing glucose and allulose is expected to result in better tolerability than allulose alone while also having superior benefits to sucrose as the glucose-allulose disaccharide does not contain fructose, which is known to be important in driving the metabolic effects of soft drinks.

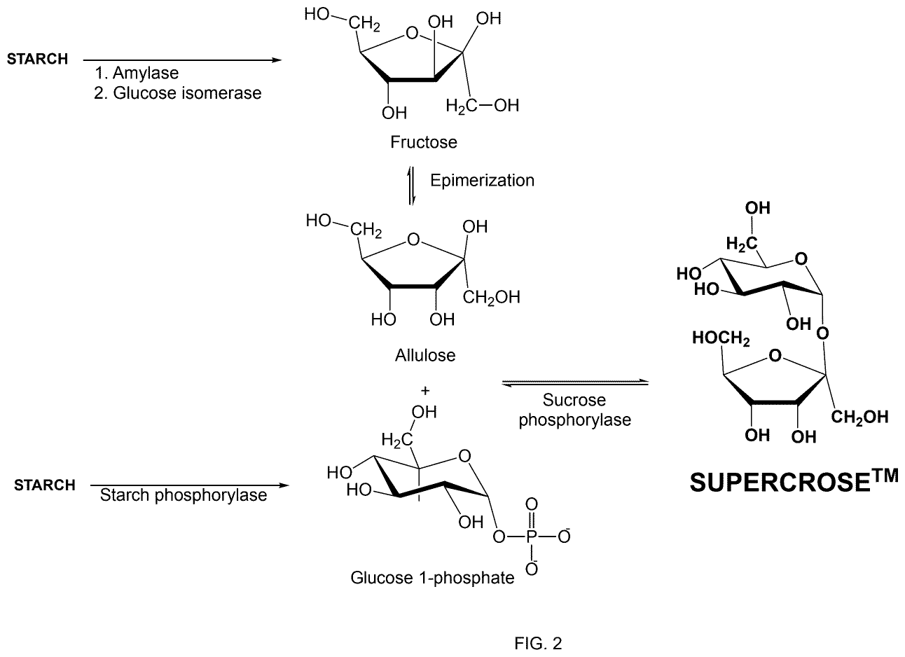

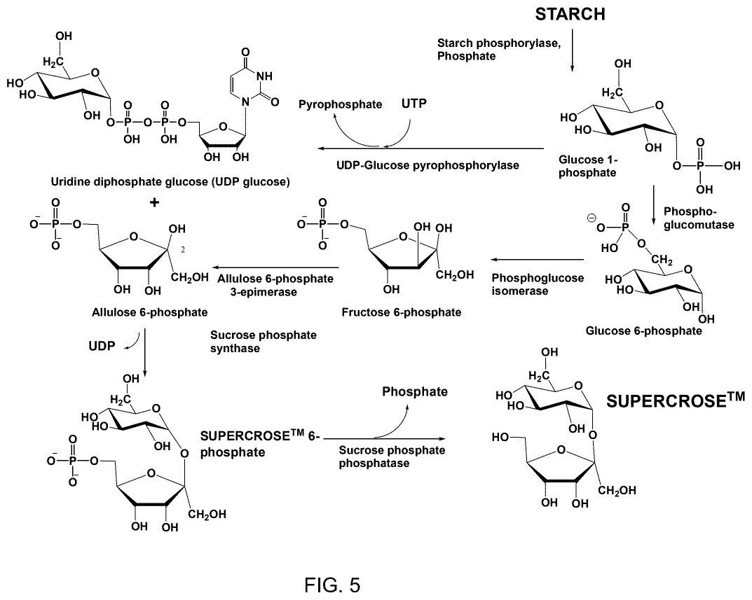

In a specific example, method embodiments for making SUPERCROSE™ can begin with starch as its initial substrate, and involve the initial production of glucose-1-phosphate from α-glucan phosphorylase (starch phosphorylase) followed by its reaction with allulose using sucrose phosphorylase (FIG. 2). This first method can also be generated in a one-pot-reaction from starch and pure allulose in the presence of α-glucan phosphorylase and sucrose phosphorylase. A second method, in one iteration, involves additional enzymatic steps beginning with starch and using an energy recycling system that may be more suitable for industrial production (FIGS. 5-7). These methods provide the glucose-allulose disaccharide (α-D-glucopyranosyl-(1→2)-β-D-allulofuranoside) herein called ‘SUPERCROSE™’ product). We also provide methods for purified glucose-allulose mixtures.

According to a more specific embodiment, provided is a method for producing SUPERCROSE™ that involves subjecting a starch sample with a starch phosphorylase in the presence of phosphate to produce glucose-1-phosphate; and subjecting allulose and the produced glucose-1-phosphate to sucrose phosphorylase under conditions to produce SUPERCROSE™ (FIG. 2). While these reactions could be run separately, it is also possible for this to be done together in the same reaction chamber. In a specific example, the steps of this method are conducted in a one-pot-reaction. As its key attributes, the process produces the disaccharide from inexpensive starch, requires no added glucose-1-phosphate, and yields the disaccharide with less than 1% contamination by sucrose. Typically, the starting starch sample is obtained from a plant source such as corn cassava, potato, other grains, or sweet potato.

In another embodiment, provided is a one-pot-reaction method in which starch, starch phosphorylase, allulose and sucrose phosphorylase are mixed in a single reaction to produce SUPERCROSE™.

According to a more specific method embodiment, provided is a method for producing SUPERCROSE™, that involves:
a. subjecting a starch sample to a glucose isomerase (e.g. xylose isomerase) under isomerizing conditions sufficient to convert one or more hexose units of the starch sample into fructose, wherein, optionally, the isomerizing conditions comprise heating the aqueous solution to 40-70 °C for a sufficient time to convert the hexose units into fructose;
b. producing allulose by subjecting the fructose produced in step (a) to an allulose epimerase under conditions such that the fructose is converted into allulose;
c. subjecting the starch sample of step (a) or a different starch sample with a starch phosphorylase in the presence of phosphate under conditions to produce glucose-1-phosphate;
d. combining the allulose of step (b) with the glucose-1-phosphate of step (c) in the presence of sucrose phosphorylase under conditions to produce SUPERCROSE™.

In yet another embodiment, and referring to FIG. 5, provided is a method for producing SUPERCROSE™ as a product that involves (a) subjecting UDP-glucose and allulose-6-phosphate to sucrose phosphate synthase under conditions to produce SUPERCROSE-6-phosphate (and UDP) and (b) subjecting the SUPERCROSE-6-phosphate to sucrose phosphate phosphatase to produce α-D-glucopyranosyl-(1→2)-β-D-allulofuranoside (SUPERCROSE™). This method may also be conducted in a one-pot-reaction.

In a specific example, UDP-glucose is produced by subjecting glucose-1-phosphate to UDP-glucose pyrophosphorylase in the presence of UTP to produce UDP-Glucose (and PPi). Moreover, optionally, the glucose-1-phosphate may be produced by subjecting starch to starch phosphorylase in the presence of phosphate to produce glucose-1-phosphate. It is noted that glucose-1-phosphate implemented in other method variations described herein may be produced from starch.

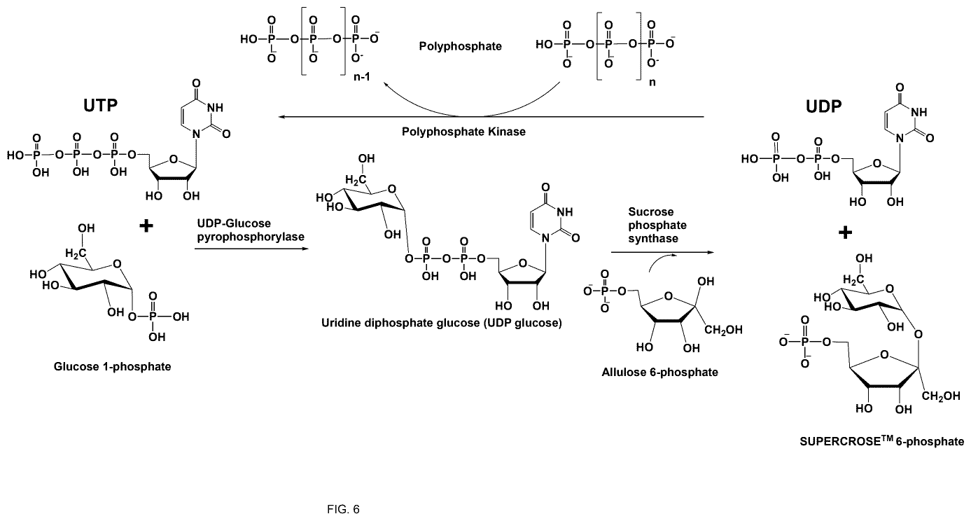

In a more specific example, UDP may be used to generate UTP by subjecting it to polyphosphate kinase (FIG. 6) in the presence of polyphosphate. Notably, UDP produced as a result of the sucrose phosphate synthase reaction (see FIG. 5) can be used for the polyphosphate kinase reaction to generate UTP. In one specific example, the polyphosphate kinase used is Cytophaga hutchinsonii polyphosphate kinase 2 (ChuPPD2). Optionally, UTP can be generated by subjecting UDP to nucleoside diphosphate kinase (NDPK) to produce UTP.

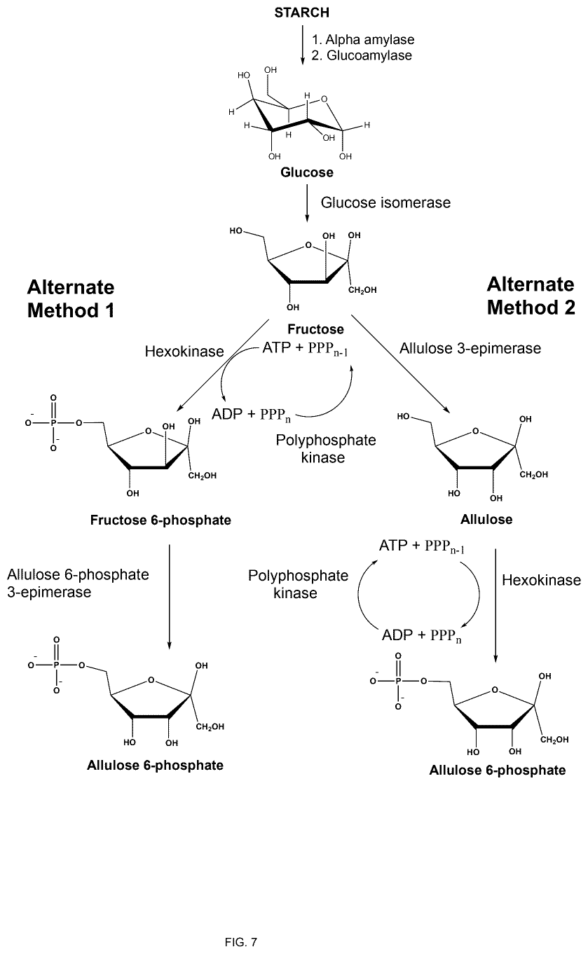

In another example, the allulose-6-phosphate described above may be produced by subjecting fructose-6-phosphate to allulose-6-phosphate-3-epimerase under conditions to produce allulose-6-phosphate (FIG. 7). Moreover, the fructose-6-phosphate may be produced by subjecting glucose-6-phosphate to phosphoglucose isomerase under conditions to produce fructose-6-phosphate; and wherein the glucose-6-phosphate may be produced by subjecting glucose-1-phosphate with phosphoglucomutase.

In an alternate example, the allulose-6-phosphate may be produced by subjecting allulose to hexokinase in the presence of ATP to produce allulose-6-phosphate and ADP. Optionally, such ADP is subjected to polyphosphate kinase in the presence of polyphosphate or hexaphosphate to produce ATP that may be used to produce additional allulose-6-phosphate (FIG. 7). In a specific example, the allulose is produced by subjecting fructose, said fructose optionally being produced from starch as a starting material, to allulose-3-epimerase to produce allulose.

In another alternate example, the allulose-6-phosphate is produced by subjecting fructose to hexokinase in the presence of ATP to produce ADP and Fructose-6-phosphate, and subjecting the fructose-6-phosphate to allulose-6-phosphate-3-epimerase to produce allulose-6-phosphate. Optionally, such ADP is subjected to polyphosphate kinase in the presence of polyphosphate or hexaphosphate to produce ATP that may be used to produce additional fructose-6-phosphate.

In certain examples, glucose in the methods described herein may be formed by adding water and α-amylase (such as, e.g. from Bacillus licheniformis) to starch, under degradation conditions to form glucose. Typically, though not necessarily, degradation of the starch to glucose is induced by increasing the temperature of the water, amylase and starch mixture, typically between approximately 60-95 °C. This will generally liquify the starch. After the subjecting the starch mixture to amylase, the mixture my further subjected to a glucoamylase (e.g. such as from Chalara paradoxa) under conditions to cause further degradation, thereby forming a glucose enriched composition. The glucose enriched composition can then be subjected to a glucose isomerase to produce fructose.

Methods of making SUPERCROSE™ described herein may further involve purifying such product using methods known in the art, such as column chromatography. Also, a non-limiting list of examples of enzyme sources and background is provided in Table 1. It is also noted that enzymatic reactions described herein may be performed in an aqueous medium that comprises one or more components such as buffer like: phosphate, carbonate, acetate, borate, citrate and Tris, MOPS; nitrogen sources and inorganic salts.

According to other embodiments, provided is a composition that includes SUPERCROSE™ and is used for increasing sweetness of a beverage or food product. Also provided is a beverage or food product comprising SUPERCROSE™.

Further embodiments pertain to a method of enhancing sweetness of a beverage or food product, the method involving adding an amount of SUPERCROSE™ to the beverage or food product, wherein the sweetness of the amount of SUPERCROSE™ is distinct from an equivalent amount of glucose or allulose. In a specific embodiment, the amount of SUPERCROSE™ comprises less calories and/or lower glycemic index compared to an equivalent amount of sucrose.

A further embodiment, pertains a mixture of glucose and allulose, wherein said mixture comprises less than 1% fructose. Also provided is a beverage or food product comprising such mixture. Moreover, provided is a method of enhancing sweetness of a beverage or food product comprising adding an amount of the mixture to the beverage or food product.

In specific examples, the concentration of starch or starch derivatives is 1-100 g/L, the amount of starch (glucan) phosphorylase is 0.1-1000 U/mL, the amount of glucose isomerase is 0.1-1000 U/mL, the amount of allulose epimerase is 0.1-1000 U/mL, the amount of the sucrose phosphorylase is 0.1-1000 U/mL.

TABLE 1
Non-limiting List of Exemplary Enzymes for use in SUPERCROSE ™ product Synthesis

| Name | Source Organism | Reference |
|---|---|---|
| Starch Phosphorylase | Zea mays | (1973), Arch. Biochem. Biophys., 156, 276- |
| | | 286 |
| | Thermus | Journal of Industrial Microbiology, vol. 24, pp. |
| | caldophilus GK₂₄ | 89 to 93, 2000. |
| | Potato | J. Biol. Chem., vol. 261, pp. 8230 to 8236, |
| | | 1986. J. Biol. Chem., vol. 266, pp. 18446 to |
| | | 18453, 1991 |
| | Arabidopsis thalia | J. Biol. Chem., vol. 261, pp. 8230 to 8236, |
| | | 1986. |
| | Thermus aquaticus | J. Appl. Glycosci., vol. 48, No. 1, pp. 71 to 78, |
| | | 2000. |
| Sucrose phosphorylase | Monilia sitophila | Current opinion in Biotechnology, 2001, vol. |
| | | 12, pp. 371 to 375. |
| | Leuconostoc mesenteroides | U.S. Pat. No. 7,968,830 |
| | Streptococcus mutans | U.S. Pat. No. 7,968,830 |
| | Lactobacillus acidophilus | U.S. Pat. No. 7,968,830 |
| | Listeria monocytogenes | U.S. Pat. No. 7,968,830 |
| | Oenococcus oeni | U.S. Pat. No. 7,968,830 |
| | Bifidobacterium adolescentis | U.S. Pat. No. 11,021,691 |
| | Agrobacterium vitis | NCBI P33910 |
| | Bifidobacterium longum | Accession no. Q84BY1 |
| | Pseudomonas putrefaciens | Accession no. AAD40317 |
| | Rhodopirellula baltica | Accession no. Q7UIS9 |
| | Shewanella baltica | Accession no. Q3Q4P1 |
| | Shewanella frigidimarina | Accession no. Q3NMD1 |
| | Solibacter usitatus | Accession no. Q43TL5 |
| | Synechococcus sp. | Accession no. O68858, Q7U3J7 |
| Epimerase | Flavonifractor plautii | U.S. Pat. Nos. 9,988,618 and 11,634,740 |
| | Agrobacterium tumefaciens | U.S. Pat. No. 8,030,035 |
| | P. cichorii | U.S. Pat. No. 10,480,018 |
| | Clostridium cellulolyticum | WO2015/032761A1 |
| | Amycolatopsis taiwanensis | U.S. Pat. No. 10,480,018 |
| | Pseudonocardia spinosispora | U.S. Pat. No. 10,480,018 |
| | Modestobacter sp | U.S. Pat. No. 10,480,018 |
| | Mesorhizobium sp. | U.S. Pat. No. 10,480,018 |
| | Bulkholderia RP64 | PCT/US16/24217 |
| Glucose isomerase (aka xylose | Multiple species | www.brenda- |
| isomerase) | provided in Brenda | enzymes.org/enzyme.php?ecno=5.3.1.5 |
| | database for EC | |
| | 5.3.1.5 | |
| Amylase | Multiple species | www.brenda- |
| | provided in Brenda | enzymes.org/enzyme.php?ecno=3.2.1.1 |
| | database for EC | |
| | 3.2.1.1 | |
| Polyphosphate Kinase 2 | Cytophaga hutchinsonii | |
| allulose-6-phosphate | E. coli | 3CT7 |
| epimerase | | |
| UDP-glucose | Helicobacter pylori | 3JUJ |
| pyrophosphorylase | | |
| phosphoglucomutase | | multiple commercially available sources |
| Phosphoglucoseisomerase | | multiple commercially available sources |
| sucrose phosphate synthase | Thermosynechococcus elongatus | 6LDQ |
| sucrose phosphate | Zea mays | Q9FQ11.1 |
| phosphatase | | |
| Sucrose phosphorylase | Bifidobacterium adolescentis | WP_011742626.1 |

For use in industrial scale processes, in certain embodiments, the above enzymes can be recombinantly expressed in an organism, such as E. coli according to techniques known in the art.

The production method can use sucrose phosphorylase and α-glucan phosphorylase in either purified or crude forms, with reactions carried out in batch or continuous formats, even when the enzymes are immobilized. As a method of immobilization, a carrier binding method (e.g. covalent binding method, ion binding method, or physical adsorbing method), a crosslinking method or an inclusion method (lattice type or microcapsule type) can be used.

An α-glucan phosphorylase is preferably derived from potato, sweet potato, Fava bean, Arabidopsis thaliana, spinach, corn, rice, wheat or a citrus. It is preferable that an α-glucan phosphorylase is derived from a type L α-glucan phosphorylase. An α-glucan phosphorylase is preferably derived from an α-glucan phosphorylase of type L, L2 or H derived from potato, type L or H derived from sweet potato, type L or H derived from Fava bean, type L or H derived from Arabidopsis thaliana, type L derived from spinach, type L derived from corn, type L or H derived from rice, type H derived from wheat, or type H derived from a citrus, is more preferably derived from an α-glucan phosphorylase of type L or L2 derived from potato, type L derived from sweet potato, type L derived from Fava bean, type L derived from Arabidopsis thaliana, type L derived from spinach, type L derived from corn, or type L derived from rice, is more preferably derived from an α-glucan phosphorylase of type L derived from potato or type L derived from Arabidopsis thaliana, and is most preferably derived from an α-glucan phosphorylase of type L derived from potato.

The process described in FIG. 2 involves using a starch (or α-glucan) phosphorylase along with a sucrose phosphorylase. α-Glucan phosphorylases are well known in the art, and can be obtained commercially from many suppliers, who provided both natural and recombinant forms of the enzyme from many sources. In one embodiment, the sucrose phosphorylase is from Bifidophulus adolescentis. Starch, or its solubilized forms, for example, corn starch, is the feedstock in one embodiment for glucose, and is introduced into the process based on its glucose equivalents. For α-glucan phosphorylases to operate, organic phosphate must be present in the buffer. Since the phosphate is recycled, only catalytic amounts are needed, in one embodiment, 1 to 10 mM. The amount of α-glucan phosphorylase contained in the solution when the reaction is initiated is typically about 0.05 to 1,000 U/grams starch, preferably about 0.1 to 500 U/grams starch, more preferably about 0.5 to 100 U/grams starch relative to starch in the solution when the reaction is initiated. If the weight of α-glucan phosphorylase is too large, the enzyme denatured during the reaction may be easily aggregated. If the amount of α-glucan phosphorylase used is too small, the yield of α-glucan may be lowered.

Those skilled in the art will appreciate that the method embodiments provided herein enable the production of a new disaccharide that is fructose free. The methods typically involve supplanting fructose as the ketohexose (i.e. sucrose) with allulose to form the disaccharide of α-D-glucopyranosyl-(1→2)-β-D-allulofuranoside (SUPERCROSE™). Moreover, it is contemplated that a different aldohexose other than glucose can be formed with allulose to create other alternative fructose-free disaccharides. For example, a disaccharide can be formed that involves combining allose and allulose, where allose is implemented in place of glucose. This and other fructose-free disaccharides are contemplated.

### Compositions Containing Glucose/Allulose Mixture or SUPERCROSE™

In other embodiments, a composition is disclosed that includes glucose and allulose, “G/A composition”. The G/A composition may contain little to no fructose, or at least not a metabolically relevant amount. The G/A composition may have less 3%, 2% or 1% fructose, by weight, or less than 5 parts relative to glucose and allulose in the G/A composition. In one embodiment, the G/A composition includes 10-70%, by weight, glucose, or 10-70 parts by weight relative to allulose. In more specific embodiments, the G/A composition includes 20-60%, or 20-60 parts, by weight, glucose. The G/A composition includes 10-70%, by weight, allulose, 10-70 parts by weight relative to glucose, and in more specific embodiments, the G/A composition includes 20-60% allulose, or 20-60 parts allulose relative to glucose.

The G/A and SUPERCROSE™ compositions may be available as a dry powder, liquid, or semi-liquid (e.g., syrup). When the G/A composition and/or SUPERCROSE™ composition is used as a food material or a food additive for alleviating abnormal carbohydrate metabolism and/or abnormal lipid metabolism, it may be in the form of a solid preparation such as a tablet, a capsule, or a powder or granules to be dissolved in beverages, etc.; a semisolid preparation such as jelly or syrup; or a high-concentration liquid solution to be diluted before use; or the like.

Products Containing a G/A Composition and/or SUPERCROSE™

Further, the G/A composition and/or SUPERCROSE™ may be suitably added to foods to give healthy diets or foods for patient, which are used for alleviating abnormal carbohydrate metabolism and/or abnormal lipid metabolism. As optional components, vitamins, carbohydrates, coloring agents, flavoring agents, and the like, which are ordinary food additives may be suitably added. The food may be taken in an arbitrary liquid or solid form. It may be encapsulated in gelatin or the like and taken as a soft capsule. Such a capsule is formed using a gelatin shell, for example, which is prepared by adding water to the raw material gelatin to dissolve, and adding a plasticizer (glycerin, D-sorbitol, etc.) thereto.

SUPERCROSE™ and/or the G/A composition can be used for the same purposes as in the case of sucrose and may also be used for cooking or in tea, coffee, seasonings (mirin (sweet rice wine for cooking), etc.), and the like.

Specific examples of food and beverage include western confectioneries (puddings, jellies, gumdrops, candies, drops, caramels, chewing gums, chocolates, pastries, butter creams, custard, cream puffs, pancakes, breads, potato chips, fried potatoes, popcorn, biscuits, crackers, pies, sponge cakes, waffles, cakes, doughnuts, biscuits, cookies, rice crackers, okoshi (millet-and-rice cakes), steamed buns, candies, etc.), dried noodles (macaroni, pasta), egg products (mayonnaise, fresh cream), beverages (functional beverages, lactic acid beverages, lactic acid bacteria beverages, thick lactic beverages, fruit juice beverages, beverages containing no fruit juice, fruit juice beverages containing pulp, clear carbonated beverages, carbonated beverages containing fruit juice, fruit-colored carbonated beverages), nonessential grocery items (green tea, black tea, instant coffee, cocoa, canned coffee beverages), dairy products (ice cream, yogurt, milk for coffee, butter, butter sauce, cheese, fermented milk, processed milk), pastes (marmalade, jam, flower paste, peanut paste, fruit paste, fruits preserved in syrup), meat products (ham, sausage, bacon, dry sausage, beef jerky, lard), seafood products (fish ham, fish sausage, boiled fish paste, chikuwa (tube-shaped fish sausage), hanpen (cake of ground fish), dried fishes, dried bonito, dried mackerel, dried sardine, sea urchin eggs, shiokara (salted and fermented fish guts) of squid, dried cuttlefish, dried fishes seasoned with mirin, dried shellfishes, smoked salmon or the like), tsukudani (foods boiled down in soy) (small fishes, shellfishes, wild vegetables, mushrooms, kelp), curries (instant curry, retort curry, canned curry), seasonings (bean paste, powdered bean paste, soy sauce, powdered soy sauce, moromi (unrefined sake or soy sauce), fish sauce, sauce, catsup, oyster sauce, bouillon cube, barbecue sauce, curry roux, stew base, powdered soup, powdered broth, paste, instant soup, furikake (dry condiment to be sprinkled over rice), dressing, salad oil), fried products (deep-fried tofu, deep-fried tofu confectioneries, instant Chinese noodles), soybean milk, margarine, and shortening.

The above food and beverage can be prepared by mixing the composition with ordinary food materials in the usual manner. The amount of the composition to be added to the above food and beverage varies depending on the kind of food, and is not limited. Usually, an amount of 0.1 to 50 wt % is preferable.

The above food and beverage can be used also as functional foods, dietary supplements, or health foods. The form thereof is not limited. Examples of food products are highly nutritious proteins with a good amino acid balance, such as milk protein, soybean protein, and ovalbumin; decomposition products thereof; albumen oligopeptide; soybean hydrolysates; mixtures of simple amino acids; and the like. They can be used in the usual manner, and may also be used in the form of soft capsules, tablets, etc.

Examples of supplements and functional foods include preparations containing saccharides, fats, trace elements, vitamins, emulsifiers, flavoring agents, etc., in the form of liquid diets, defined formula diets, elementary diets, health beverages, capsules, enteral nutrients, or the like. Among the above-mentioned food products, beverages such as an isotonic beverage, or a nutrition supplement beverage, for example, may further contain, in order to improve the nutritional balance and flavor thereof, nutritional additives such as amino acids, vitamins, and minerals, sweeteners, spices, flavoring agents, coloring agents, and the like.

### EXAMPLES

### Example 1: Enzymatic Process for Producing α-D-Glucopyranosyl-(1→2)-β-D-Allulofuranoside (SUPERCROSE™) Using the Sucrose Phosphorylase Method

### Sucrose Phosphorylase Cloning, Expression and Immobilization

A DNA gBlock encoding the gene of E. coli gene of sucrose phosphorylase from Bifidobacterium adolescentis containing the amino acid replacements: Q331E; R393N; D445P; D446T; Q460E; E485H was obtained from IDT (SEQ ID NO: 1). This variant was previously reported (Cerdobbel A, et al (2011) Increasing the thermostability of sucrose phosphorylase by a combination of sequence- and structure-based mutagenesis. Protein Eng Des Sel. 24, 829-34). The DNA was resuspended at 10 ng/μL and digested for 8 h at 37 °C with restriction enzymes NcoI and NheI and cloned on vector pJExpress414 (DNA 2.0). The resulting plasmid was used to express the sucrose phosphorylase (SEQ ID NO: 5). in E. coli C43(DE3) (37 °C, overnight growth, IPTG 0.5 mM) in LB media. The next morning, cells were pelleted (0.8 g) and resuspended in 5 mL of BugBuster® master mix (Novagen) and mixed gently for 20 minutes at room temperature. The lysate was pelleted in 1.5 mL tubes, at 21,000×g for 5 minutes and the supernatant was mixed with NiNTA resin (1.5 mL of resuspended resin washed and resuspended in 4 mL of Lysis buffer: Tris HCl, 20 mM pH 8.0; NaCl 500 mM, imidazole 10 mM). The lysate and the resin were then incubated and gently spun at 4 °C for 1 h.

The lysate-resin was then loaded on a column and washed with: (a) 2 mL wash: Tris, 20 mM; NaCl, 1 M; followed by (b) 1 mL wash: Tris, 20 mM; NaCl, 150 mM; followed by (c) 2 mL wash: Tris, 20 mM; NaCl, 500 mM; imidazole, 20 mM; and (d) eluted with elution buffer: 1 mL Tris HCl, 10 mM, pH 8.0, NaCl 250 mM, imidazole 500 mM. Buffer was exchanged by ultracentrifugation (AMICON, 15 mL, 50,000 MW cutoff) to 40 mM phosphate buffer, pH 7.0.

### Immobilization in Beads

The enzyme sucrose phosphorylase was incubated at room temperature in 40 mM phosphate buffer, pH 7.0 with agitation in a rotisserie. Using about 50 mL of ReliZymenHFA403 beads. Immobilization has been shown to increase the heat stability of the Sucrose Phosphorylase from B. adolescentis (Cerdobbel A, et al, (2010) Increasing the thermostability of sucrose phosphorylase by multipoint covalent immobilization. J Biotechnol. 150, 125-30).

SUPERCROSE™ is then produced by reacting at 40 °C equimolar amounts of allulose and glucose 1-phosphate in the presence of sucrose phosphorylase attached to beads while adding dropwise a solution of 10% of barium acetate (FIG. 2).

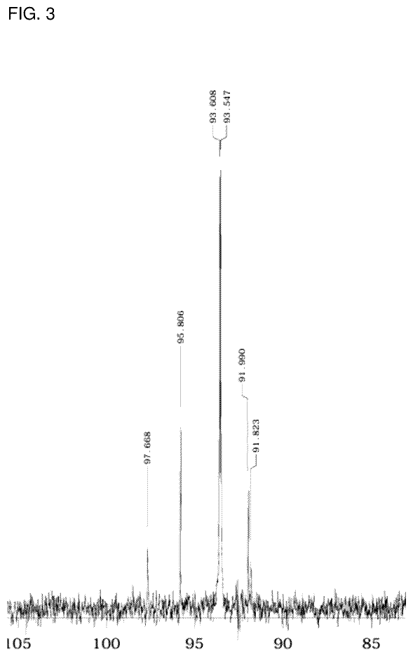

FIG. 3 shows the ¹³C-NMR spectrum of SUPERCROSE™ in D20 prepared using sucrose phosphorylase. This reaction uses a labeled ¹³C-labeled glucose 1-phosphate. After overnight reaction the peaks at 91.990 and 91.823 emerge.

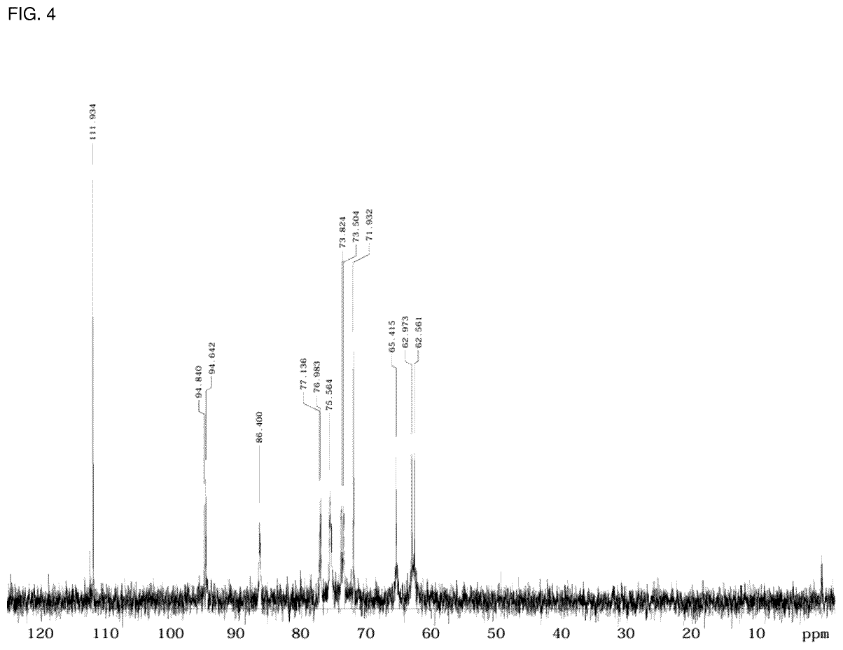

SUPERCROSE™ was also made chemically and the ¹³C-NMR spectrum is shown in FIG. 4 and was confirmed as the same spectrum pattern as noted for SUPERCROSE™ by Morimoto et al. (2015) Production and application of a rare disaccharide using sucrose phosphorylase from Leuconostoc mesenteroides'. J. Bioscience Bioengineering 119, 652-656. The NMR spectrum in FIG. 3 is also consistent with this spectral pattern, as the difference in ppm comes from the absence of the 0 ppm signal. Since that latter experiment has enriched ¹³C in the labeled glucose-1-phosphate, the other signals are dwarfed since the natural abundance of ¹³C is only 1%.

### Example 2: Enzymatic Process for Producing α-D-Glucopyranosyl-(1→2)-(3-D-Allulofuranoside (SUPERCROSE™) Using Sucrose Phosphate Synthase Method

Phosphorylase a (2.4.1.1, rabbit muscle, 25 U/mg) was obtained from Sigma-Aldrich and used without further purification. Other phosphorylases from other suppliers and other organisms may also be used.

UDP-glucose pyrophosphorylase cloning and expression. A DNA gBlock encoding the gene of UDP-glucose phosphorylase was obtained from IDT (SEQ ID NO: 2). The DNA was resuspended at 10 ng/μL and digested for 8 h at 37 °C with restriction enzymes NcoI and NheI and cloned on vector pJExpress414 (DNA 2.0). The resulting plasmid was used to express the UDP-glucose pyrophosphorylase in E. coli C43(DE3) 37 °C, overnight growth, induction with IPTG 0.5 mM in LB media. The E. coli expressed a protein (SEQ ID NO: 6). The next morning, cells were pelleted (0.8 g) and resuspended in 5 mL of BugBuster® master mix (Novagen) and mixed gently for 20 minutes at room temperature. The lysate was pelleted in 1.5 mL tubes, at 21,000 xg for 5 minutes and the supernatant was mixed with NiNTA resin (1.5 mL of resuspended resin washed and resuspended in 4 mL of Lysis buffer Tris HCl, 20 mM pH 8.0; NaCl 500 mM, imidazole 10 mM). The lysate and the resin were then incubated and gently mixed at 4 °C for 1 h.

The lysate-resin mix was then loaded on a column and washed with: (a) 2 mL wash: Tris, 20 mM; NaCl, 1 M; followed by (b) 1 mL wash: Tris, 20 mM; NaCl, 150 mM; followed by (c) 2 mL wash: Tris, 20 mM; NaCl, 500 mM; imidazole, 20 mM; and (d) eluted with elution buffer: 1 mL Tris HCl, 10 mM, pH 8.0, NaCl 250 mM, imidazole 500 mM. Buffer was exchanged by ultracentrifugation (AMICON, 15 mL, 50,000 MW cutoff) to 40 mM phosphate buffer, pH 7.0.

Sucrose phosphate synthase cloning and expression. A DNA gBlock encoding the gene of sucrose phosphate synthase was obtained from IDT (SEQ ID NO: 3). The DNA was resuspended at 10 ng/μL and digested for 8 h at 37 °C with restriction enzymes NcoI and NheI and cloned on vector pJExpress414 (DNA 2.0). The resulting plasmid was used to express the sucrose phosphate synthase in E. coli C43(DE3) 37 °C, overnight growth, induction with IPTG 0.5 mM in LB media. The E. coli expressed a protein (SEQ ID NO: 7). The next morning, cells were pelleted (0.8 g) and resuspended in 5 mL of BugBuster® master mix (Novagen) and mixed gently for 20 minutes at room temperature. The lysate was pelleted in 1.5 mL tubes, at 21,000 xg for 5 minutes and the supernatant was mixed with NiNTA resin (1.5 mL of resuspended resin washed and resuspended in 4 mL of Lysis buffer Tris HCl, 20 mM pH 8.0; NaCl 500 mM, imidazole 10 mM). The lysate and the resin were then incubated and gently mixed at 4 °C for 1 h.

The lysate-resin mix was then loaded on a column and washed with: (a) 2 mL wash: Tris, 20 mM; NaCl, 1 M; followed by (b) 1 mL wash: Tris, 20 mM; NaCl, 150 mM; followed by (c) 2 mL wash: Tris, 20 mM; NaCl, 500 mM; imidazole, 20 mM; and (d) eluted with elution buffer: 1 mL Tris HCl, 10 mM, pH 8.0, NaCl 250 mM, imidazole 500 mM. Buffer was exchanged by ultracentrifugation (AMICON, 15 mL, 50,000 MW cutoff) to 40 mM phosphate buffer, pH 7.0.

Sucrose Phosphate Phosphatase (SPP) cloning and expression. A DNA gBlock encoding the gene of SPP was obtained from IDT (SEQ ID NO: 4). The DNA was resuspended at 10 ng/μL and digested for 8 h at 37 °C with restriction enzymes NcoI and NheI and cloned on vector pASK-IBA43plus. The resulting plasmid was used to express the sucrose phosphate phosphatase (SPP) in E. coli C43(DE3) (37 °C, overnight growth, induction with anhydrotetracycline (0.67 μg/mL) in LB media. The E. coli expressed a protein (SEQ ID NO: 8). The next morning, cells were pelleted (0.8 g) and resuspended in 5 mL of BugBuster® master mix (Novagen) and mixed gently for 20 minutes at room temperature. The lysate was pelleted in 1.5 mL tubes, at 21,000×g for 5 minutes and the supernatant was mixed with NiNTA resin (1.5 mL of resuspended resin washed and resuspended in 4 mL of Lysis buffer Tris HCl, 20 mM pH 8.0; NaCl 500 mM, imidazole 10 mM). The lysate and the resin were then incubated and gently mixed at 4 °C for 1 h.

The lysate-resin mix was then loaded on a column and washed with: (a) 2 mL wash: Tris, 20 mM; NaCl, 1 M; followed by (b) 1 mL wash: Tris, 20 mM; NaCl, 150 mM; followed by (c) 2 mL wash: Tris, 20 mM; NaCl, 500 mM; imidazole, 20 mM; and (d) eluted with elution buffer: 1 mL Tris HCl, 10 mM, pH 8.0, NaCl 250 mM, imidazole 500 mM. Buffer was exchanged by ultracentrifugation (AMICON, 15 mL, 50,000 MW cutoff) to 40 mM phosphate buffer, pH 7.0

Hexokinase reaction. A mixture of allulose (49.012 mg/mL); polykinase (1 μM); sodium hexametaphosphate (1 mM); ATP (1 mM) in MOPS buffer (50 mM) and MgCl₂ (10 mM); 100 units of hexokinase. Incubated at 37 °C for 12 h.

Multistep Reaction (with Three Enzymes)

Uridine triphosphate (UTP), 90 μmol; 13C-1 glucose 1-phosphate, 90 μmol, hexokinase reaction (348 pmol of allulose). Combined with hexokinase reaction to a final volume of 2 mL. added 20 μL of each of the following enzymes1: UDP-Glucose pyrophosphorylase, sucrose phosphate synthase and sucrose phosphate phosphatase. The enzymes were in buffer Tris buffer 20 m M, pH 7.5 to: 10 mM Tris, pH 8.0; 100 mM KCl; 10 mM EDTA; 0.5% Tween 20; 0.5% Triton X-100; 50% glycerol. Incubated at 37 °C for 3 days. Uridine triphosphate (UTP), 90 μmol; 13C-1 glucose 1-phosphate, 90 μmol, hexokinase reaction (348 pmol of allulose). Combined with hexokinase reaction to a final volume of 2 mL. added 20 μL of each of the following enzymes¹: UDP-Glucose pyrophosphorylase, sucrose phosphate synthase and sucrose phosphate phosphatase. The enzymes were in buffer Tris buffer 20 m M, pH 7.5 to: 10 mM Tris, pH 8.0; 100 mM KCl; 10 mM EDTA; 0.5% Tween 20; 0.5% Triton X-100; 50% glycerol. Incubated at 37 °C for 3 days. ¹ The enzymes have been purified using His-tag, however they co-elute with other proteins. Attempts to purify have used sephadex to separate them by size exclusion. However it might be more beneficial to do the SEC before the His tag purification.

Multistep Reaction (with Three Enzymes) in Presence of Polyphosphate.

Similar to the reaction described above but with the presence of 1 mM PolyP₆ (Sodium hexametaphosphate, Sigma), and 1 mM Cytophaga hutchinsonii Polyphosphate Kinase 2 (PPK2).

### Example 3 Assessing Sweetness of Supercrose™ and Allulose-Glucose (AG) Mixtures

The sweetness of α-D-glucopyranosyl-(1→2)-β-D-allulofuranoside (also known as SUPERCROSE™) has not been reported, and so whether it is sweet and the level of sweetness has not been assessed. Sweetness is mediated in part by the level of activation of the sweet taste receptors of the tastebuds of the tongue, and hence the sweetness of a disaccharide might be quite different from the monosaccharides as the disaccharide is converted to monosaccharides by invertases and/or sucrases which are present in the gut.

We used a relative sweetness score in which a 10% solution of sucrose was given a score of 1.0 (Trumbo P R, et al (2021). Perspective: Measuring Sweetness in Foods, Beverages, and Diets: Toward Understanding the Role of Sweetness in Health. Adv Nutr. 12:343-354). A variety of sugars were tested and confirmed the observation that fructose (10%) is sweeter than sucrose (mean 1.15), as was a 50:50 mixture of fructose and glucose (10%, mean 1.23) while both glucose and allulose (10%) solutions were less sweet than sucrose (mean of 0.68 and 0.75, respectively). However, and unexpectedly, a 50:50 allulose-glucose (AG) mixture (10%) was sweeter than either glucose or allulose alone (mean 0.88) while SUPERCROSE™ (10%, 99% pure) tended to be less sweet (mean, 0.6). Using the National Institute of Health category scaling method for taste, three sugars were strongly sweet (score>7), including fructose-glucose (8.25), fructose (7.87 and sucrose (7), three sugars were moderately sweet (mean score 5 to 7), including 50:50 AG mix (6.5), allulose (6.1) and glucose (5.2) while the SUPERCROSE™ was only mildly sweet (score<5) (with a score of 4.8). These studies provide the unexpected observation that the AG mix was sweeter than expected compared to the known sweetness of allulose and glucose, while SUPERCROSE™ tended to be less sweet than expected.

### Example 4: Chemical Synthesis of SUPERCROSE™

Provided below is the step-wise chemical synthesis process of producing SUPERCROSE™:

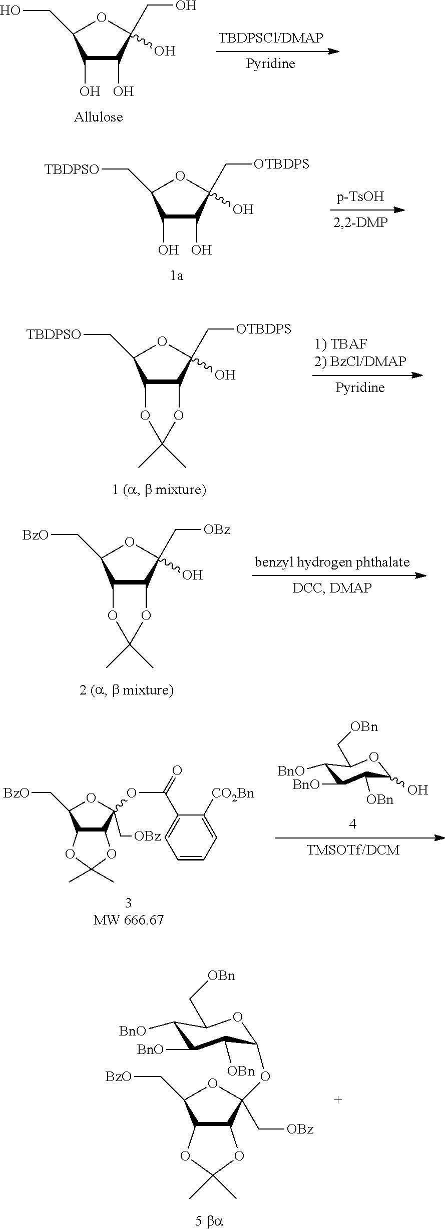

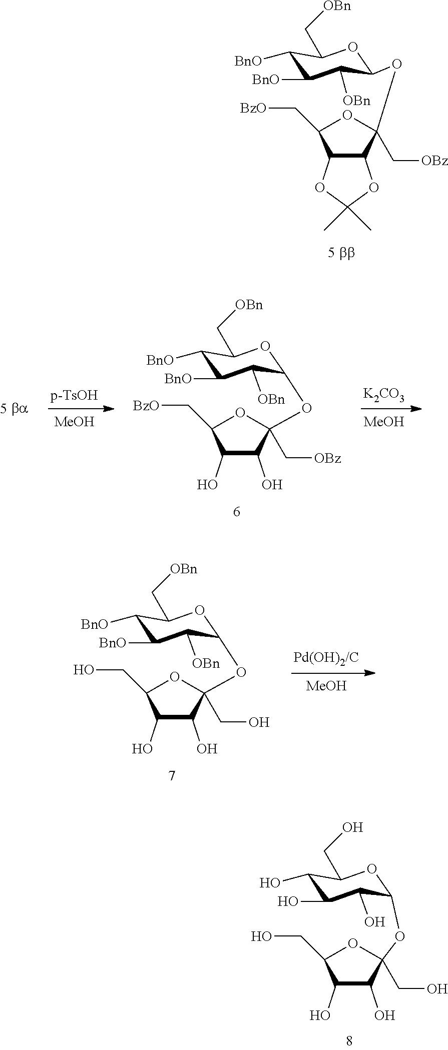

To a solution of allulose (20 g, 111 mmol) in pyridine (600 mL) was added tert-butyldiphenylchlorosilane (TBDPSCl; 87 mL, 333 mmol) and 4-(dimethylamino)pyridine (DMAP; 2.72 g, 22.2 mmol) at room temperature and the resultant mixture was stirred at room temperature for 22 h (2:20 pm). After quenching the reaction by adding water (20 ml), the reaction mixture was evaporated to dryness. The residue was co-evaporated with toluene (2×), then EtOAc (700 ml) and cold water (500 ml) was added (pH of the water phase was 2-3. Caution! Do not to neutralize it. Otherwise, pyridine from HCl salt will be released out to cause difficulty for column). The aqueous phase extracted with EtOAc (300 ml). The combined organic extracts were washed with water (300 ml) and brine (200 ml). After drying over anhydrous MgSO₄ and concentration under vacuum, the residue was passed through a short plug of silica gel (n-hexane, then 20% EtOAc in n-hexane) to give crude 1,6-di-O-(tert-butyldiphenylsilyl) D-tagatofuranose Ia as an off-white sticky semisolid (71 g). It may contain a small amount of pyridine.

A solution of the above crude triol Ia (71 g) in 2,2-dimethoxypropane (1000 mL) was stirred at room temperature in the presence of p-TsOH·H₂O (1.1 g, 5.6 mmol) for 1.5 h (2:35 m), TLC show almost no reaction. 4 g of p-TsOH·H₂O and the mixture stirred at rt overnight. TLC indicated the completion of reaction. 50 ml of sat. NaHCO₃ was added to quench reaction. After stirring a while, the mixture was transferred into a separatory funnel, diluted with hexane/EtOAc (1:1, 500 ml), washed with water (200 ml) and brine (100 ml). The aqueous phase extracted with EtOAc (200 ml). The extract was washed brine (50 ml), The combined organics were dried over Na₂SO₄. After evaporation of solvents and further drying on the lyophilize, 70.9 g of the product obtained as off-white sticky oil. HNMR shows it's quite pure.

To a solution of 1 (70.9 g, 111 mmol) in THF (700 mL) was added tetrabutylammonium fluoride (TBAF; 1 M in THF, 222 mL) solution at room temperature (2:00 pm). After stirring for 20 h at room temperature, Dowex 50 W×8 (138 g), CaCO₃ (46.3 g), MeOH (3255 mL) were added to the reaction mixture, which was further stirred for 1 h at room temperature. The resultant suspension was filtered through a Celite pad and the filter cake was washed with MeOH (200 ml). After concentration in vacuo, the crude 3,4-O-isopropylidene-D-tagatofuranose was co-evaporated with pyridine (2×100 ml), toluene (150 ml) and used for the next step without further purification.

To a solution of the above triol and DMAP (6.78 g, 55.5 mmol) in pyridine (292 mL)/CH₂Cl₂ (1684 mL) was added a solution of benzoyl chloride (BzCl; 38.3 mL, 330 mmol) in DCM (50 ml) dropwise at 0 °C (9:08 am) over 50 min. and the reaction mixture was stirred for 1 h at room temperature. The reaction mixture was quenched by adding drop wise MeOH (130 mL, with cooling) and stirred for further 20 min. After removal of solvents, the residue was partitioned into 1 M HCl aq and EtOAc layers. After extraction of aqueous layer by EtOAc twice, combined organic layers were washed with water followed by brine, and dried over anhydrous MgSO4. The crude material was purified by flash column chromatography on silica gel (20% EtOAc in n-hexane) to afford dibenzoate 2 (34.3 g, 70% in two steps, a/b=3:1) as a colorless oil.

To a solution of 2 (10.77 g, 25.13 mmol) in DCM (~300 mL) was added benzyl hydrogen phthalate (22.6 g, 87.98 mmol), DCC (15.6 g, 75.41 mmol) and DMAP (3.1 g, 25.13 mmol) at 0 °C (9:00 am). The reaction mixture was stirred at 0 °C for 10 min, then cooling bath removed. Stirring continued over night at room temperature (20 h). The reaction mixture was filtered through a Celite pad and the filtrate was washed successively with water (200 ml), 5% NaHCO₃ (200 ml×2), and brine (100 ml). The organic layer was dried over Na₂SO₄. Upon evaporation of solvents, the residue was purified by flash column chromatography on silica gel to give the glycosyl donor (14.2 g, 84.8%).

46 g of product obtained from another batch starting with 30.7 g of starting material 2.

A solution of compound 3 (3.0 g) and 2,3,4,6-tetra-O-benzyl-D-glucopyranose (3.65 g, 6.75 mmol, 1.5 eq) in anhydrous dichloromethane (~80 ml) was cooled in acetonitrile dry ice bath. To this solution was added drop wise TMSOTf (1.23 ml) at −45 °C. The reaction mixture was stirred at −40 to −30 °C for 30 min. Sat. NaHCO₃ (50 ml) was added to the mixture to quench the reaction. 100 ml of DCM was added. The mixture was transferred into separatory funnel to get layers. The organic layer was washed with water (1×) and brine (1×), dried over Na₂SO₄. After evaporation of solvent, the residue was purified by flash column eluting with hexane/EtOAc gradient (95:5 to 6:1) to give two anomers 5 bb (1.03 g pure, 0.67 g less pure, 39.8%) and 5 ba (1.7 g, 39.8%).

7.13 g of product obtained from other batches.

A mixture of compound 5 ba (1.7 g) in methanol (25 ml) was added p-TsOH at rt. The reaction mixture was stirred at rt for 45 h. TLC shows a small amount of SM left. The reaction flask was put on rotavapor and stirred at 30 °C water bath for 1 h. TLC shows no change. Et₃N (1 ml) was added. After stirring for a while, the mixture was evaporated to dryness. The residue was re-dissolved in EtOAc (50 ml), washed with sat aqueous NaHCO₃, brine. The aqueous was extracted with CHCl₃. The combined organic extracts were dried over Na₂SO₄ and concentrated to dryness, and then purified by silica gel flash chromatography eluted with hexane/EtOAc gradient (up to 2:1) to give 6 (834 mg) in 50.9% yield. Colorless syrup.

A mixture of 6 (830 mg, 0.911 mol) and K₂CO₃ (1.256 mg, 0.5 mmol) in MeOH (110 mL) was stirred for 1 h at room temperature. The reaction mixture was filtered through a Celite pad (1 cm) and concentrated under vacuum. The crude material was purified by flash column chromatography on silica gel eluted with hexane/EtOAc gradient (2:1 to 1:3) to afford tetraol 7 (0.427 g, 66.7%). Note: before vacuum concentrated of solvent, the filtrate should be neutralized.

To a suspension of compound 7 (290 mg, 0.4 mmol) in MeOH (5 mL) and water (40 ml) was added 10% Pd(OH)₂ on carbon (105 mg). The suspension was degassed followed by refilling hydrogen 3 times and then stirred under hydrogen atmosphere (1 atm, with balloon) at 40 °C overnight (24 h). TLC showed the completion of Rx. Cooled to rt, the reaction mixture was filtered through celite pad and rinsed with water (2×). The filtrate was extracted with EtOAc (2×), DCM (2×) and Et₂O to give a slightly colored clear aqueous solution. Attempt to decolor by treatment with charcoal at 40 °C did not work out. Then this solution was concentrated under vacuum to dryness. The residue was further dried on lyophilize to give the desired product as off-white solid (135 mg, 99%).

What is claimed is:

1. A method for producing α-D-glucopyranosyl-(1→2)-β-D-allulofuranoside, the method comprising
a. subjecting a starch sample with a starch phosphorylase in the presence of phosphate under conditions to produce glucose-1-phosphate; and
b. subjecting the glucose-1-phosphate of step (a) together with allulose to sucrose phosphorylase under conditions to produce d-glucosyl-d-alluloside.

2. The method of claim 1, wherein steps (a) and (b) are conducted in a single reaction container.

3. (canceled)

4. (canceled)

5. (canceled)

6. A method for producing α-D-glucopyranosyl-(1→2)-β-D-allulofuranoside, the method comprising
a. subjecting a starch sample to an amylase and a glucose isomerase under isomerizing conditions sufficient to convert one or more hexose units derived from the starch sample into fructose, wherein, optionally, the isomerizing conditions comprise heating the aqueous solution to 40-70 °C for a sufficient time to convert the one or more hexose units into fructose;
b. producing allulose by subjecting the fructose produced in step (a) to an allulose epimerase under conditions such that the fructose is converted into allulose;
c. subjecting the starch sample of step (a) or a different starch sample with a starch phosphorylase in the presence of phosphate under conditions to produce glucose-1-phosphate;
d. combining the allulose of step (b) with the glucose-1-phosphate of step (c) in the presence of sucrose phosphorylase under conditions to produce d-glucosyl-d-alluloside.

7. (canceled)

8. The method of claim 6 wherein any of steps (a)-(d) are performed in an aqueous medium that comprises one or more components selected from the group consisting of phosphate, carbonate, acetate, borate, citrate and tris, alcohols such as methanol and ethanol, esters such as ethyl acetate, ketones such as acetone, amides such as acetoamide, and natural or synthetic medium containing carbon sources, nitrogen sources and inorganic salts.

9. (canceled)

10. (canceled)

11. A method for producing of α-D-glucopyranosyl-(1→2)-β-D-allulofuranoside as a product, the method comprising (a) subjecting UDP-glucose and allulose-6-phosphate to sucrose phosphate synthase under conditions to produce SUPERCROSE-6-phosphate and (b) subjecting the SUPERCROSE-6-phosphate to sucrose phosphate phosphatase to produce α-D-glucopyranosyl-(1→2)-β-D-allulofuranoside.

12. The method of claim 12, wherein steps (a) and (b) are conducted in a single reaction container.

13. The method of claim 11, wherein UDP-glucose is produced by subjecting glucose-1-phosphate to UDP-glucose pyrophosphorylase in the presence of UTP to produce UDP-Glucose.

14. The method of claim 13, wherein the glucose-1-phosphate is produced by subjecting starch to starch phosphorylase in the presence of phosphate to produce glucose-1-phosphate.

15. The method of claim 13, wherein UTP is produced from UDP by subjecting UDP to polyphosphate kinase in the presence of polyphosphate to produce UTP.

16. The method of claim 11, wherein the allulose-6-phosphate is produced by subjecting fructose-6-phosphate to allulose-6-phosphate-3-epimerase to produce allulose-6-phosphate.

17. The method of claim 16, wherein the fructose-6-phosphate is produced by subjecting glucose-6-phosphate to phosphoglucoseisomerase to produce fructose-6-phosphate; and wherein the glucose-6-phosphate is produced by subjecting glucose-1-phosphate with phosphoglucomutase.

18. The method of claim 11, wherein the allulose-6-phosphate is produced by subjecting allulose to hexokinase in the presence of ATP to produce allulose-6-phosphate and ADP, wherein, optionally, the ADP is subjected to polyphosphate kinase in the presence of phosphate to produce ATP that may be used to produce further allulose-6-phosphate.

19. The method of claim 18, wherein the allulose is produced by subjecting fructose, said fructose optionally being produced from starch as a starting material, to allulose-3-epimerase to produce allulose.

20. The method of claim 11, wherein the allulose-6-phosphate is produced by subjecting fructose to hexokinase in the presence of ATP to produce ADP and fructose-6-phosphate, and subjecting the fructose-6-phosphate to allulose-6-phosphate-3-epimerase to produce allulose-6-phosphate.

21. (canceled)

22. A beverage or food product comprising α-D-glucopyranosyl-(1→2)-β-D-allulofuranoside produced by the method of claim 11.

23. A method of enhancing sweetness of a beverage or food product, the method comprising adding an amount of α-D-glucopyranosyl-(1→2)-β-D-allulofuranoside produced by claim 11 to the beverage or food product, wherein the sweetness of the amount of α-D-glucopyranosyl-(1→2)-β-D-allulofuranoside is greater than an equivalent amount of glucose or allulose.

24. The method of claim 23, wherein the amount of α-D-glucopyranosyl-(1→2)-β-D-allulofuranoside comprises less calories and/or less glycemic index compared to an equivalent amount of sucrose.

25. (canceled)

26. (canceled)

27. (canceled)

28. The method of claim 11, wherein any of the subjecting steps are performed in an aqueous medium that comprises one or more components selected from the group consisting of phosphate, carbonate, acetate, borate, citrate and tris, alcohols such as methanol and ethanol, esters such as ethyl acetate, ketones such as acetone, amides such as acetoamide, and natural or synthetic medium containing carbon sources, nitrogen sources and inorganic salts.

29. The method of claim 11, wherein the α-D-glucopyranosyl-(1→2)-β-D-allulofuranoside is isolated from other reagents by a purification method.

30. The method of claim 29, wherein the purification method is column chromatography.
